(19) United States
(12) Patent Application Publication
(10) Pub. No.: US 2009/0210055 A1
(43) Pub. Date: Aug. 20, 2009

(54) ARTIFICIAL OPTIC NERVE NETWORK MODULE, ARTIFICIAL RETINA CHIP MODULE, AND METHOD FOR FABRICATING THE SAME

(75) Inventors: Tao-Chih Chang, Taoyuan County (TW); Min-Lin Lee, Hsinchu City (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(73) Assignee: INDUSTRIAL TECHNOLOGY RESEARCH INSTITUTE, Hsinchu (TW)

(21) Appl. No.: 12/211,829

(22) Filed: Sep. 17, 2008

(30) Foreign Application Priority Data
Feb. 19, 2008 (TW) 97105776

(51) Int. Cl. A61F 2/14

(52) U.S. Cl. 623/6.63

(57) ABSTRACT

An artificial retina chip module including a signal processing chip, a first polymer bump layer, and a photodiode array chip is provided. The signal processing chip includes a plurality of first pad disposed on a surface thereof. The first polymer bump layer includes a plurality of polymer bumps insulated from one another. Each of the first polymer bumps is composed of a polymer material and a conductive layer coated on the polymer material. Each first polymer bump is embedded into the corresponding first pad and the signal processing chip, wherein one end of the first polymer bump protrudes from the first pad and the other end thereof protrudes from a back surface of the signal processing chip. The photodiode array chip is disposed at one side of the signal processing chip and is electrically connected to the signal processing chip through the first polymer bumps.

### CROSS-REFERENCE TO RELATED APPLICATION

This application claims the priority benefit of Taiwan application serial no. 97105776, filed on Feb. 19, 2008. The entirety of the above-mentioned patent application is hereby incorporated by reference herein and made a part of specification.

### BACKGROUND OF THE INVENTION

1. Field of the Invention

The present invention generally relates to an artificial optic nerve network module, an artificial retina chip module and a method for fabricating the same, and more particularly, to an artificial optic nerve network module and an artificial retina chip module which utilize flip chip bonding technique for electrical connecting different chips, and a method for fabricating the same.

2. Description of Related Art

In the past, ophthalmology could do little about diseases related to retina pathological changes, such as macular degeneration, and retinitis pigmentosa (RP). Conventional technologies are used to strengthen remaining vision with optical aids, such as magnifying glass, and telescope.

Recently, electronic eye becomes a new and hot ophthalmic research field. The principle of the electronic eye is to capture optical information of an ambient image, and then transfer the optical information into an electronic signal by a camera, an image processor, and a photo-electronic signal converting process. The electronic signal is then transmitted to an implant inside an eye. The implant decodes the electronic signal and releases a certain type of corresponding current to stimulate the remaining retina nerve cells and thus triggering the vision. Recently, many researchers put great effort in the research of substituting light with electricity in the field. Specifically, there had been tried to stimulate with electricity at where the nerve fibers concentrate in the transmitting path of the vision, including retina, optic nerves, and cortex of occipital lobe.

Currently, there are many countries and enterprises involved in the development of the electronic eye. For example, an artificial retina researching group co-funded by Massachusetts Institute of Technology and Harvard University (MIT-Harvard) has developed a artificial retina structure.

FIGS. 1A and 1B are schematic diagrams illustrating a structure of an artificial electronic eye developed by the artificial retina researching group of MIT-Harvard, and a chip and electrode plate portion of the structure, respectively. The artificial retina researching group of MIT-Harvard designs an electronic interface with the computer chip technology in developing the artificial electronic eye. Referring to FIG. 1A, an electronic eye 100 includes an 820-nm, fixed-direction laser power source 110, and a micro charge coupled device (CCD) video camera 120 which may output an amplitude adjustable laser. The video camera 120 includes a signal processing micro chip which converts visional information into electronic codes transmitted by the laser beam. The power source 110 and the video camera 120 are all embedded in a sunglass 200. Referring to FIG. 1B, an artificial retina 300 implanted in a human body includes a photodiode plate 310, a flexible plate 320, and a signal processing chip 330. The photodiode plate 310 and the signal processing chip 330 enclose to hold one end of the flexible plate 320. The other end of the flexible plate 320 is attached to the retina and includes electrodes 322 for stimulating the retina. The photodiode plate 310 is adapted for processing a light signal, and the signal processing chip 330 is adapted for converting the light signal into an electronic signal and generating a suitable signal to stimulate the optic nerve cells. When illuminating the photodiode plate 310, the laser beam generates a power source and initiates the signal processing chip 330. The signal processing chip 330 then instructs the electrodes 322 on the other end of the flexible plate 320 to generate a current. Such an artificial retina 300 is attached to a fore-end of the original retina for initiating the epi-retina cell to generate visional signals and then transmits the visional signals to the optic nerves and the visional cortex.

In the current artificial optic retina 300, the photodiode plate 310 and the signal processing chip 330 are typically electrically connected by wire bonding. However, as the amount of the electrode arrays (pixels) increasing, the conventional wire bonding technique is not sufficient for matching the increment of the I/O number. Further, the signal transmittance by using the wire bonding technique for electrical connection may encounter the problems of a lower transmission rate and incapable of real-time transmission.

### SUMMARY OF THE INVENTION

Accordingly, the present invention is directed to an artificial optic nerve network module. The artificial optic nerve network module uses biocompatible and flexible polymer bumps serving as electrical contacts for connecting different chips to replace the conventional metal electrodes. This arrangement may avoid the injuries to the retina caused by the rigid metal electrodes having no elasticity when the eyeball turns suddenly.

The present invention is further directed to an artificial retina chip module and a method for fabricating the same. The present invention utilizes the flip chip bonding technique for electrically connecting different chips, so as to solve the problem of the conventional technology that cannot be used for those chips having a large I/O number and is not adapted for real-time transmission because of the wire bonding processed used thereby.

The present invention is also directed to a method for fabricating flexible electrodes on a chip. With the steps of drilling holes, forming a conductive layer, coating and patterning a polymer layer, etc., the present invention is adapted to form a plurality of flexible polymer bump on the chip.

The present invention provides an artificial optic nerve network module. The artificial optic nerve network module mainly includes a plurality of chips and at least one polymer bump layer. The chips are adapted for generating an artificial vision and are stacked on one another. The polymer bump layer is embedded in one of the chips, so as to electrically connect the chip with the adjacent chip. The polymer bump layer includes a plurality of polymer bumps insulated from one another. Each of the polymer bumps is composed of a polymer material and a conductive layer coated on the polymer material, and protrudes from an upper surface and a lower surface of the chip.

The present invention further provides an artificial retina chip module. The artificial retina chip module includes a signal processing chip, a first polymer bump layer, and a photodiode array chip. The signal processing chip includes a plurality of first pads disposed on a surface of the signal processing chip. The first polymer bump layer includes a plurality of polymer bumps insulated from one another. Each of the first polymer bumps is composed of a polymer material and a conductive layer coated on the polymer material. Each first polymer bump is embedded into the corresponding first pad and the signal processing chip, such that one end of each of the first polymer bumps protrudes from the first pad, and the other end of each of the first polymer bumps protrudes from a back surface of the signal processing chip. The photodiode array chip is disposed at one side of the signal processing chip and is electrically connected to the signal processing chip through the first polymer bumps.

The present invention further provides a method for fabricating flexible electrodes on a chip. The method comprises the following steps. First, a chip having a plurality of pads disposed on a surface thereof is provided. Then, a photo resist layer is formed on the surface of the chip for covering the pads. Next, a plurality of micro holes are formed, wherein the micro holes pass through the photo resist layer and the pads and extend inside the chip. Then, a first conductive layer is formed on the photo resist layer and the micro holes. Next, the photo resist layer is removed. A photosensitive polymer layer is formed on the surface of the chip, wherein the photosensitive polymer layer covers the pads and fills each of the micro holes. Then, the photosensitive polymer layer is patterned to form a plurality of polymer bumps. A second conductive layer is formed on a surface of each of the polymer bumps, and the second conductive layer is electrically connected to the pad. Finally, the chip is thinned, so that one end of each of the polymer bumps protrudes from the chip.

The present invention further provides a method for fabricating an artificial retina chip module. The method comprises the following steps. First, a signal processing chip having a plurality of pads disposed on a surface thereof is provided. Then, a photo resist layer is formed on the surface of the signal processing chip for covering the pads. Next, a plurality of micro holes is formed. Each of the micro holes passes through the photo resist layer and the pads, and extends inside the signal processing chip. Then, a first conductive layer is formed on the photo resist layer and the micro holes. The photo resist layer is removed. Next, a photosensitive polymer layer is formed on the surface of the chip, wherein the photosensitive polymer layer covers the pads and fills each of the micro holes. Then, the photosensitive polymer layer is patterned to form a plurality of polymer bumps. A second conductive layer is formed on a surface of each of the polymer bumps, and the second conductive layer is electrically connected to the pad. Then, the chip is thinned, so that one end of each of the polymer bumps protrudes from the chip. Finally, a photodiode array chip is provided, and the signal processing chip is electrical connected with the photodiode array chip through the polymer bumps.

The present invention forms a plurality of flexible polymer bumps on the chip by the using steps of drilling holes, forming a conductive layer, coating and patterning a polymer layer, etc. In such a way, the present invention utilizes the polymer bumps serving as electrical contacts instead of the conventional technology which using the wire bonding technique for electrically connecting the photodiode plate and the signal processing chip. This manner may solve the problem that the wire bonding technique cannot be applied to chips having a large I/O number and achieve real-time transmission.

Besides, the present invention employs a three-dimensional chip stack technology with the flexible polymer bumps made of the biocompatible polymer material to connect the signal processing chip and the photodiode array chip for miniaturization. This may provide a solution to the insufficient flexibility of the conventional artificial retinas and the risk of injuries to the retina caused by the rigid metal electrodes when the eyeball turns suddenly.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIGS. 1A and 1B are schematic diagrams illustrating a structure of an artificial electronic eye developed by the artificial retina researching group of MIT-Harvard, and a chip and electrode plate portion of the structure, respectively.

FIGS. 2A through 2J are schematic, cross-sectional diagrams illustrating the process flow for fabricating an artificial retina chip module according to an embodiment of the present invention.

FIGS. 3A through 3C are schematic, cross-sectional diagrams illustrating the process flow for packaging the artificial retina chip module according to another embodiment of the present invention.

FIG. 4 is schematic, cross-sectional diagram showing an artificial retina chip module according to another embodiment of the present invention.

FIG. 5 is a schematic, cross-sectional diagram showing an artificial optic nerve network module according to an embodiment of the present invention.

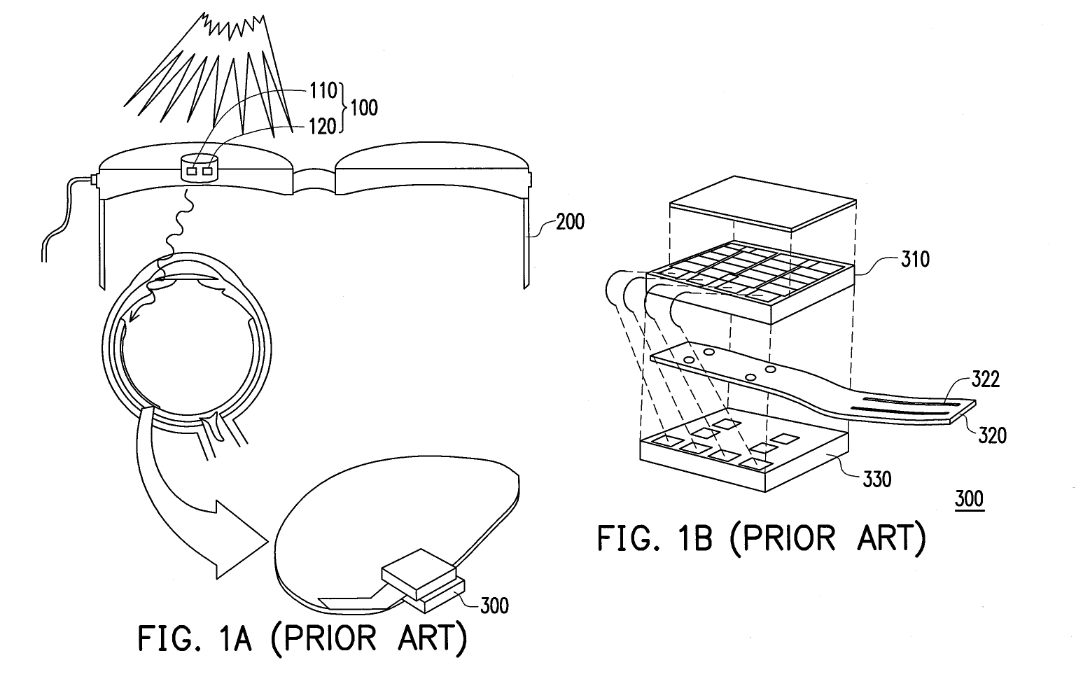

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

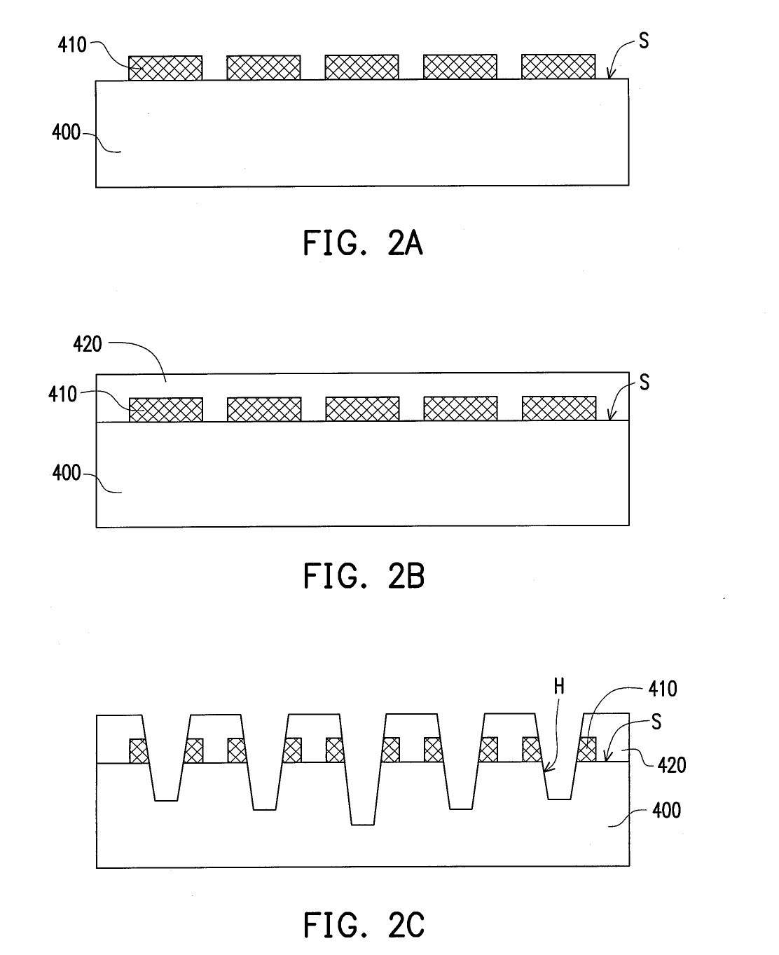

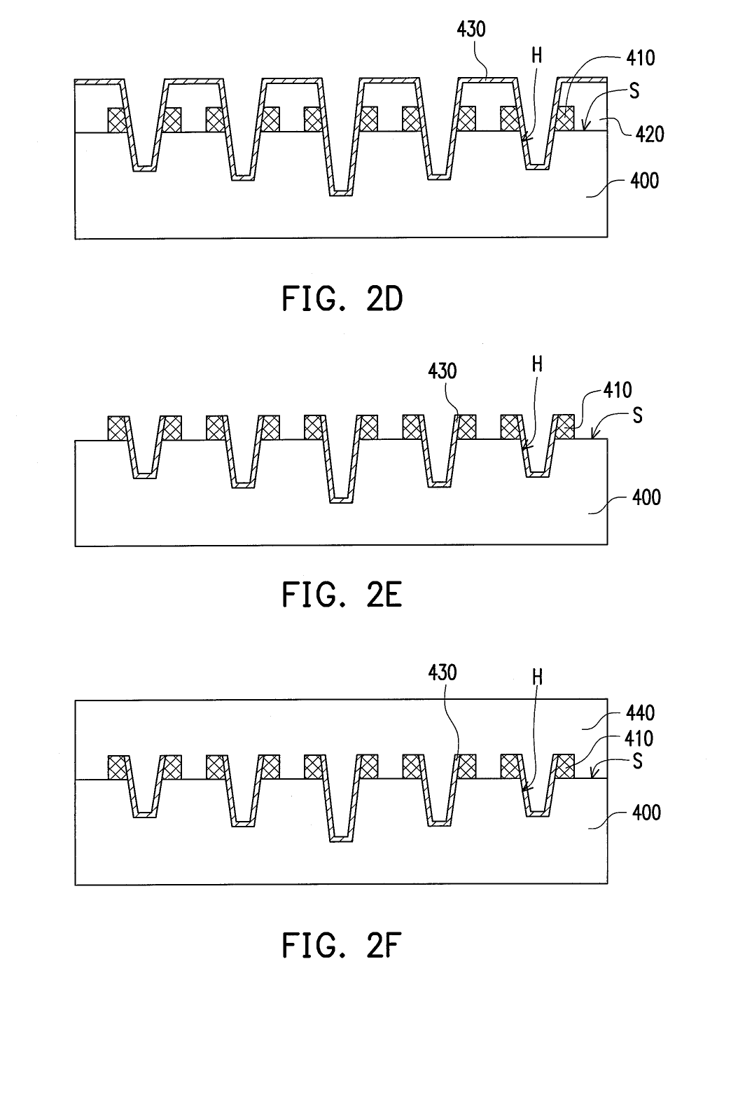

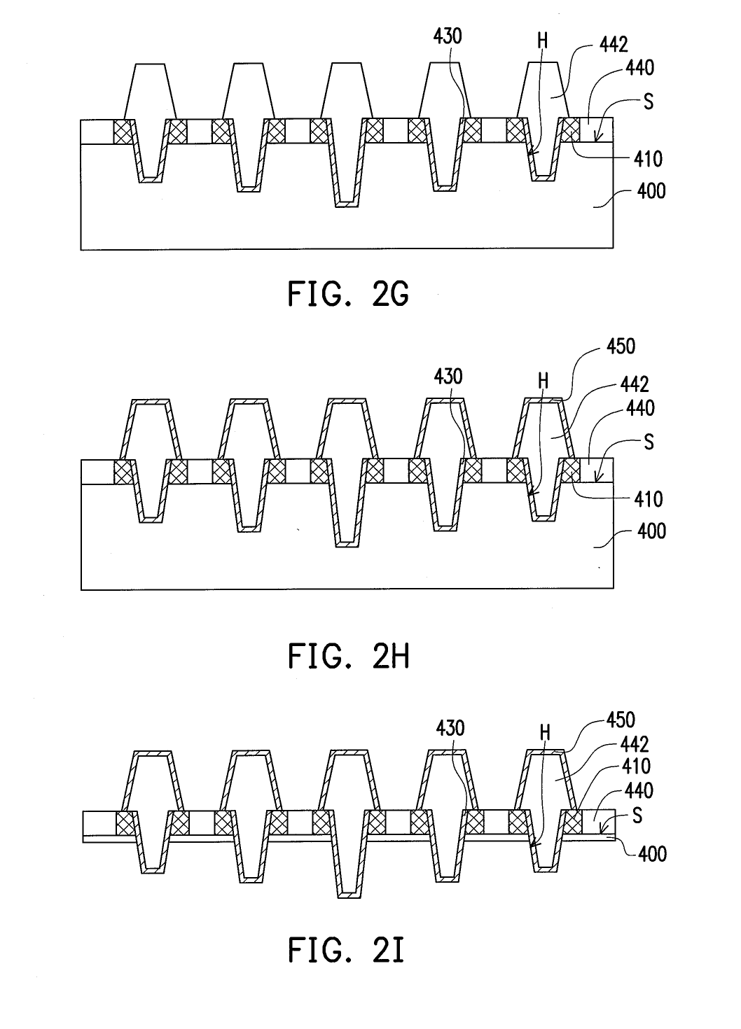

FIGS. 2A through 2J are schematic, cross-sectional diagrams illustrating the process flow for fabricating an artificial retina chip module according to an embodiment of the present invention. First, please refer to FIG. 2A, a signal processing chip 400 is provided for converting a light signal into an electronic signal, and outputting a suitable signal for stimulating optic nerves. The signal processing chip 400 includes a plurality of pads 410 disposed on a surface S thereof. As shown in FIG. 2B, a photo resist layer 420 is formed on the surface S of the signal processing chip 400 for covering the pads 410.

Then, as shown in FIG. 2C, a plurality of micro holes H is formed. Each of the micro holes H passes through the photo resist layer 420 and one of the pads 410 and extends inside the signal processing chip 400. In this step, the micro holes H may be formed by a drilling process, e.g., laser drilling, or a dry etching process. Furthermore, the depths of the micro holes H would affect the height of the polymer bumps subsequently formed on the signal processing chip 400, while these polymer bumps would be connected to the optic nerves (such as a retina). Therefore the depth H of each of the micro holes H should be varied according to the real curvature of the retina to be treated.

Then, referring to FIG. 2D, a first conductive layer 430 is formed on the photo resist layer 420 and the micro holes 430. According to one embodiment of the present invention, the first conductive layer 430 is preferably made of a biocompatible conductive material, a non-metallic conductive material, or other suitable conductive materials. Further, the biocompatible conductive material is preferred to be selected from the group consisting of titanium, gold, platinum, and their oxides. The non-metallic conductive material is preferred to be selected from the group consisting of iridium oxide and graphite. Then, as shown in FIG. 2E, the photo resist layer 420 is removed. In the step, an organic solvent can be used to remove the photo resist layer 420. In the meantime, the first conductive layer 430 on the photo resist layer 420 is also removed, while remain the pads 410 and the first conductive layer 430 on the pads 410.

Then, referring to FIG. 2F, a photosensitive polymer layer 440 is formed on the surface S of the signal processing chip 400. The photosensitive polymer layer 440 is to be subsequently processed to form the polymer bumps embedded into the signal processing chip 400. In this step, a biocompatible photosensitive polymer material, such as polyimide (PI), polydimethylsiloxane (PDMS), may be coated on the signal processing chip 400 by using a spinning coating process. As shown in FIG. 2F, the photosensitive polymer layer 440 covers on the surface S of the signal processing chip 400 and fills each of the micro holes H. Then, referring to FIG. 2G, the photosensitive polymer layer 440 is patterned to form a plurality of polymer bumps 442. In this embodiment, an exposure process, a development process, and so on are performed on the photosensitive polymer layer 440 to form the polymer bumps 442. As shown in FIG. 2G, the polymer material that filled among the pads 410 is remained for insulating the pads 410 from one another.

Then, referring to FIG. 2H, a second conductive layer 450 is formed on a surface of the polymer bumps 442. The second conductive layer 450 is electrically connected with the pads 410. In such a way, the polymer bumps 442 are electrically connected with the pads 410 through the first conductive layer 430 and the second conductive layer 450 coated on the polymer pads 442. Similarly, the second conductive layer 450 is preferably made of a biocompatible conductive material, a non-metallic conductive material, or other suitable conductive material. Further, the biocompatible conductive material is preferred to be selected from the group consisting of titanium, gold, platinum, and their oxides. The non-metallic conductive material is preferred to be selected from the group consisting of iridium oxide and graphite.

Then, referring to FIG. 2I, the signal processing chip 400 is thinned, such that one end of each of the polymer bumps 442 protrudes from the signal processing chip 400. In this embodiment, a reactive ion etching process may be performed on a backside of the signal processing chip 400 for thinning the signal processing chip 400. In such a way, the polymer bumps 442 having a first conductive layer 430 coated thereon are exposed to form the flexible polymer electrodes. Finally, as shown in FIG. 2J, a photodiode array chip 500 is provided, and one end of each of the polymer bumps 442 is connected to a corresponding electrode (not shown) of the photodiode array chip 500. Therefore, the signal processing chip 400 is electrically connected with the photodiode array chip 500 through the polymer bumps 442. Thus far, the artificial retina chip module 600 is formed according to the above processes.

Further, as shown in FIG. 2J, a biocompatible polymer layer 440′ matching the shape of the retina may be optionally formed on the back surface of the signal processing chip 400. The biocompatible polymer layer 440′ is filled between the polymer bumps 442, while exposing a bottom of each polymer bump 442. The biocompatible polymer layer 440′ can be made of a material selected from the group consisting of parylene, polyimide, polymethylmethacrylate acrylic (PMMA), chitin, chitosan, polylactic acid (PLA), polyhydroxyalkanoate (PHA), or other suitable materials. Further, the bottom of each of the polymer bumps 442 is connected to an optic nerve, usually connected to a retina, for transmitting an electronic signal to an epi-retina or a sub-retina connected thereto.

Referring to FIG. 2J, the artificial retina chip module 600 of the present invention mainly comprises a signal processing chip 400, a polymer bump layer 442a having a plurality of polymer bumps 442, and a photodiode array chip 500. The signal processing chip 400 comprises a plurality of pads 410 disposed on a surface S of the signal processing chip 400. The polymer bump layer 442a comprises a plurality of polymer bumps 442 insulated from one another. Each of the polymer bumps 442 is composed of a polymer material and a conductive layer coated on the polymer material. Further, each of the polymer bumps 442 is embedded into the corresponding pad 410 and the signal processing chip 400, so that one end of the polymer bump 442 protrudes from the pad 410 and the other end of the polymer bump 442 protrudes from a back surface of the signal processing chip 400. The photodiode array chip 500 is disposed at one side of the signal processing chip 400 and is electrically connected with the signal processing chip 400 through the polymer bumps 442. The material for fabricating the artificial retina chip module 600 has been discussed before, and it is not repeated herein.

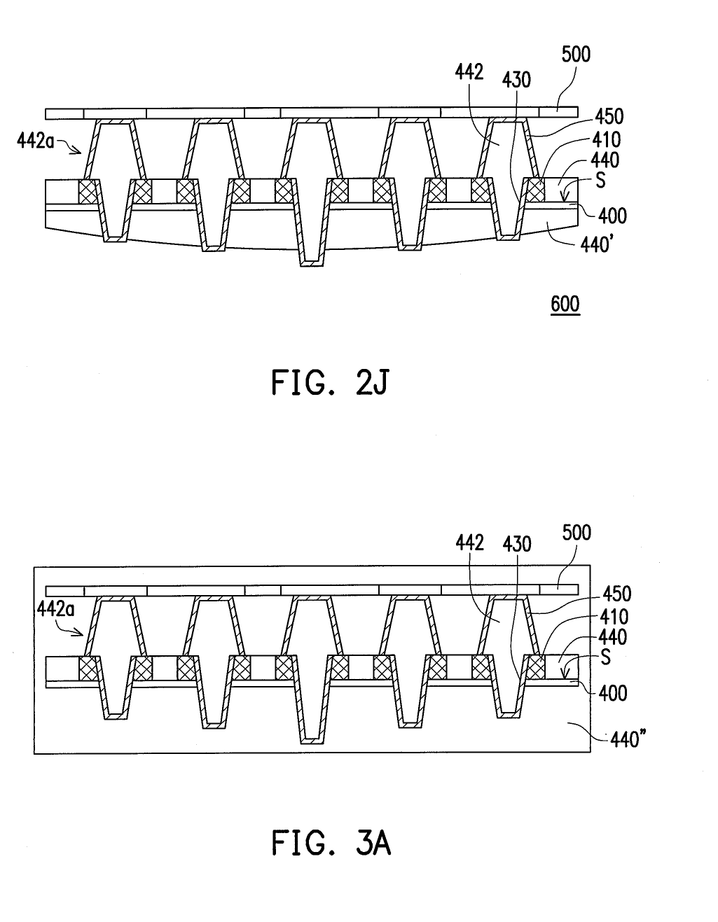

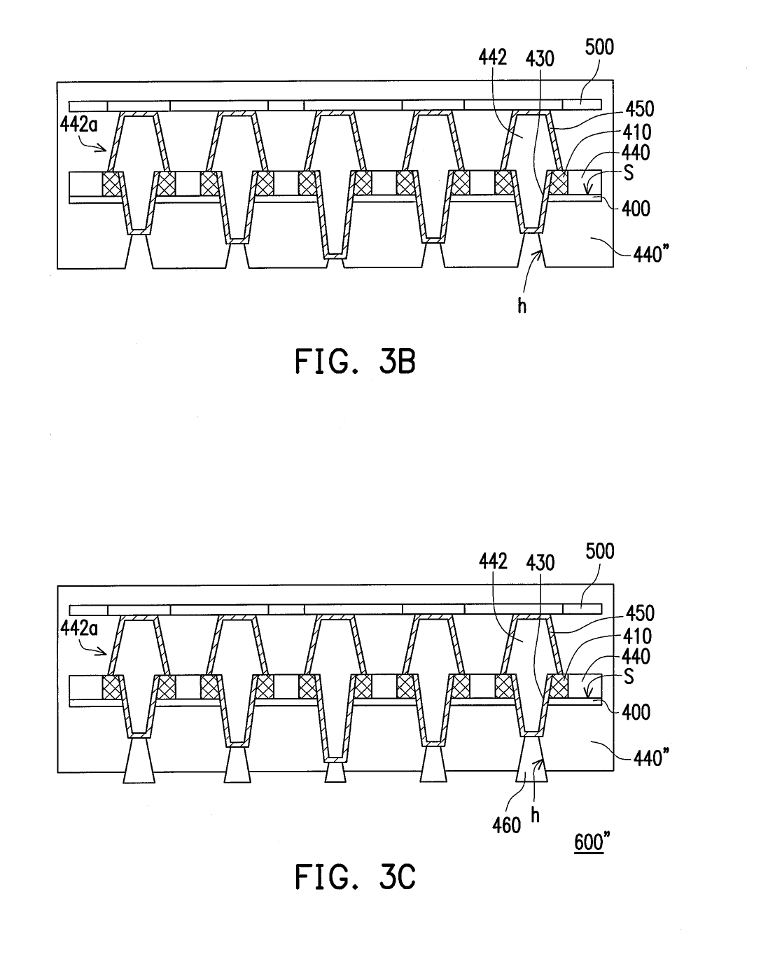

Besides forming the biocompatible polymer layer 440′ matching the shape of the retina on the back surface of the signal processing chip 400, another method for packaging the artificial retina chip module may also be used. FIGS. 3A through 3C are schematic, cross-sectional diagrams illustrating the process flow for packaging the artificial retina chip module according to another embodiment of the present invention. Referring to FIG. 3A, after the steps shown in FIGS. 2A-2I for electrically connecting the signal processing chip 400 and the photodiode array chip 500 are performed, a biocompatible polymer material 440″ is formed. The biocompatible polymer material 440″ covers the signal processing chip 400, the polymer bump layer 442a, and the photodiode array chip 500 to form a hermetic package. Further, the biocompatible polymer material 440″ can be made of parylene, polyimide, polymethylmethacrylate acrylic (PMMA), chitin, chitosan, polylactic acid (PLA), polyhydroxyalkanoate (PHA), or other suitable materials. Then, referring to FIG. 3B, a plurality of blind holes h are formed in the biocompatible polymer material 440″ for exposing the corresponding polymer bumps 442 respectively. According to an embodiment of the present invention, the blind holes h may be formed by a drilling process. Then, referring to FIG. 3C, a biocompatible conductive material is formed in each of the blind holes h. The biocompatible conductive material serves as an electrode 460 for electrically connecting with the optic nerves. This hermetic package may prevent the artificial retina chip module 600″ from being eroded by body fluid. Besides, the biocompatible polymer material 440″ covering the signal processing chip 400 provides flexibility for the artificial retina chip module 600″. Thus, the polymer bumps 442 of the signal processing chip 400 may have the same lengths and also be made of metallic materials. The lengths and the material of the polymer bumps 442 are not limited in the present invention.

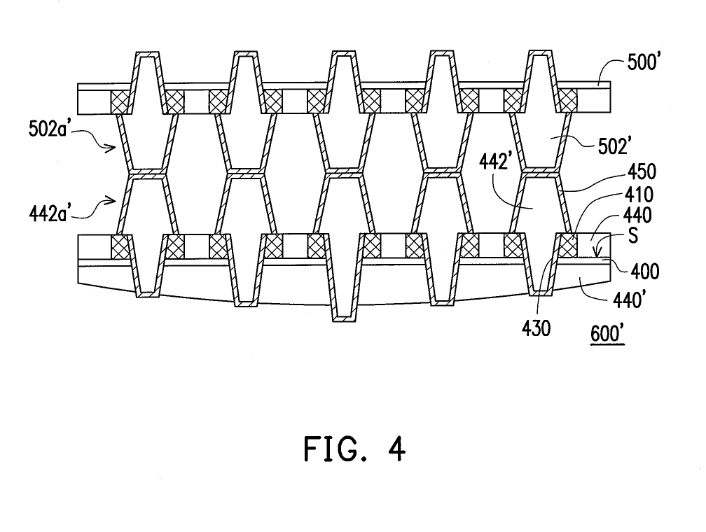

In another hand, the foregoing method for fabricating the flexible polymer electrodes can be applied for not only the signal processing chip 400, but also the photodiode array chip 500. FIG. 4 is schematic, cross-sectional diagram showing an artificial retina chip module according to another embodiment of the present invention. The structure of the artificial retina chip module 600′ is similar to that of the artificial retina chip module 600 as shown in FIG. 2J. However, the difference therebetween is that the photodiode array chip 500′ also has polymer bumps for electrically connecting with the signal processing chip 400 in the artificial retina chip module 600′. As shown in FIG. 4, the signal processing chip 400 includes a first polymer bump layer 442a, and the first polymer bump layer 442a comprises a plurality of first polymer bumps 442′ electrically insulated from one another. The structure and material for the signal processing chip 400 and the first polymer bumps 442′ have been discussed before, and it is not repeated herein. The photodiode array chip 500′ includes a second polymer bump layer 502a′. The second polymer bump layer 502a′ includes a plurality of second polymer bumps 502′ insulated from one another. The second polymer bumps 502′ may be electrical connected to the first polymer bumps 442′ by local heating, e.g., microwave bonding.

Further, the foregoing method for fabricating the flexible polymer bumps 442 on the signal processing chip 400 can be applied not only to the signal processing chip 400 and the photodiode array chip 500′, but also to other kinds of chips, e.g., biochips, for forming the flexible polymer bumps the chips.

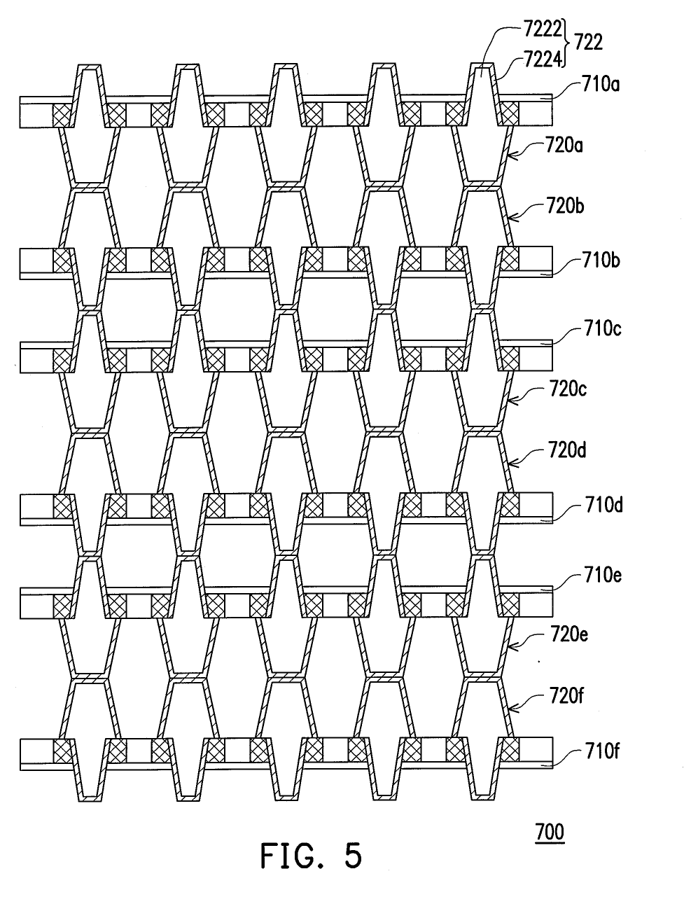

Furthermore, the foregoing polymer bump layer can also be employed in an artificial optic nerve network module for providing an electrical connection between chips. FIG. 5 is a schematic, cross-sectional diagram showing an artificial optic nerve network module according to an embodiment of the present invention. Referring to FIG. 5, the artificial optic nerve network module 700 includes a plurality of chips 710a through 710f stacked on one another for generating an artificial vision. According to an embodiment of the present invention, the chips 710a through 710f are a photodiode array chip, a signal processing chip, a chip for replacing photoreceptor cells, a chip for replacing horizontal cells, a chip for replacing bipolar cells, and a chip for replacing ganglion cells, respectively. The artificial vision can be obtained by a combination of the chips 710a through 710f. A plurality of polymer bump layers 720a through 720f are embedded into the chips 710a through 710f, respectively, so as to electrically connecting the chips 710a through 710f and the adjacent chips 710a through 710f. Each of the polymer bump layers 720a through 720f is composed of a plurality of polymer bumps 722 insulated from one another. In more details, each of the polymer bumps 722 is composed of a polymer material 7222 and a conductive layer 7224 coated on the polymer material 7222, and the polymer bumps 722 protrude from an upper surface and a lower surface of the chips 710a through 710f.

The biocompatible polymer 440′ of FIG. 2J or the biocompatible polymer material 440″ of FIG. 3C can also be applied to the artificial optic nerve network module 700 for connecting the artificial optic nerve network module 700 with the retina to be treated.

In summary, the artificial retina chip module and the artificial optic nerve network module of the present invention utilize the flip chip bonding technique with the polymer bumps made of the flexible polymer material for electrically connecting the photodiode plate and the signal processing chip in order to replace the conventional wire bonding technique. This manner may solve the problem that the wire bonding technique cannot be applied to chips having a large I/O number and achieve real-time transmission.

Besides, the present invention employs a three-dimensional chip stack technology with the flexible polymer bumps made of the biocompatible polymer material to connect the signal processing chip and the photodiode array chip for miniaturization. This may provide a solution to the insufficient flexibility of the conventional artificial retinas and the risk of injuries to the retina caused by the rigid metal electrodes when the eyeball turns suddenly.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An artificial optic nerve network module, comprising:
a plurality of chips, adapted for generating an artificial vision, and being stacked on one another; and
at least one polymer bump layer, embedded in one of the chips, so as to electrically connect the chip with another chip adjacent thereto, wherein the polymer bump layer comprises a plurality of polymer bumps insulated from one another, each of the polymer bumps is composed of a polymer material and a conductive layer coated on the polymer material, and the polymer bumps protrude from an upper surface and a lower surface of the chip.

2. The artificial optic nerve network module according to claim 1, wherein the chips at least comprises a photodiode array chip, a signal processing chip, a chip for replacing photoreceptor cells, a chip for replacing horizontal cells, a chip for replacing bipolar cells, and a chip for replacing ganglion cells.

3. The artificial optic nerve network module according to claim 1, wherein the conductive layer is made of a biocompatible conductive material or a non-metallic conductive material.

4. The artificial optic nerve network module according to claim 3, wherein the biocompatible conductive material is selected from the group consisting of titanium, gold, platinum, and their oxides.

5. The artificial optic nerve network module according to claim 3, wherein the non-metallic conductive material is selected from the group consisting of iridium oxide and graphite.

6. The artificial optic nerve network module according to claim 3, wherein a height of each of the polymer bumps varies according to a real curvature of the retina to be treated.

7. The artificial optic nerve network module according to claim 1, further comprising a biocompatible polymer layer disposed on the chip adjacent to the retina to be treated, wherein the biocompatible polymer layer exposes the polymer bumps which are connected to the retina.

8. The artificial optic nerve network module according to claim 1 further comprising a biocompatible polymer material covering the chips and the polymer bump layer, and exposing the polymer bumps which are connected to the retina to be treated.

9. An artificial retina chip module, comprising:
a signal processing chip, comprising a plurality of first pad disposed on a surface of the signal processing chip;
a first polymer bump layer, comprising a plurality of first polymer bumps insulated from one another, each of the first polymer bumps being composed of a polymer material and a conductive layer coated on the polymer material, wherein each of the first polymer bumps is embedded into the corresponding first pad and the signal processing chip, so that one end of the first polymer bump protrudes from the first pad, and the other end of the first polymer bump protrudes from a back surface of the signal processing chip; and
a photodiode array chip, disposed on one side of the signal processing chip and electrically connected to the signal processing chip through the first polymer bumps.

10. The artificial retina chip module according to claim 9, wherein the conductive layer is made of a biocompatible conductive material or a non-metallic conductive material.

11. The artificial retina chip module according to claim 10, wherein the biocompatible conductive material is selected from the group consisting of titanium, gold, platinum, and their oxides.

12. The artificial retina chip module according to claim 10, wherein the non-metallic conductive material is selected from the group consisting of iridium oxide and graphite.

13. The artificial retina chip module according to claim 9, wherein the signal processing chip further comprises a polymer material disposed among the first pads for insulating the first pads from one another.

14. The artificial retina chip module according to claim 9, wherein the first polymer bumps have different heights, and the heights of the first polymer bumps vary according to a real curvature of the retina to be treated.

15. The artificial retina chip module according to claim 9, further comprising a biocompatible polymer layer disposed at a back surface of the signal processing chip and exposing the first polymer bumps connected to the retina to be treated.

16. The artificial retina chip module according to claim 9, further comprising a biocompatible polymer material covering the signal processing chip, the first polymer bump layer, and the photodiode array chip, and exposing the first polymer bumps connected to the retina to be treated.

17. The artificial retina chip module according to claim 9, wherein the photodiode array chip comprises:
a plurality of second pads, disposed on a surface of the photodiode array chip; and
a second polymer bump layer, comprising a plurality of second polymer bumps which are insulated from one another, each of the second polymer bumps being composed of a polymer material and a conductive layer coated on the polymer material, wherein each of the second polymer bumps is embedded into the corresponding second pad and the photodiode array chip, so that one end of the second polymer bump protrudes from the second pad, and the other end of the second polymer bump protrudes from a back surface of the photodiode array chip, and each of the second polymer bumps is electrically connected to the corresponding first polymer bump.

18. The artificial retina chip module according to claim 17, wherein the conductive layer is made of a biocompatible conductive material or a non-metallic conductive material.

19. The artificial retina chip module according to claim 18, wherein the biocompatible conductive material is selected from the group consisting of titanium, gold, platinum, and their oxides.

20. The artificial retina chip module according to claim 18, wherein the non-metallic conductive material is selected from the group consisting of iridium oxide and graphite.

21. The artificial retina chip module according to claim 17, wherein the photodiode array chip further comprises a polymer material, disposed among the second pads for insulating the second pads from one another.

22. A method for fabricating a flexible electrode on a chip, comprising:
providing a chip having a plurality of pads disposed on a surface of the chip;
forming a photo resist layer on the surface of the chip for covering the pads;
forming a plurality of micro holes, wherein each of the micro holes passes through the photo resist layer and the pads, and extends inside the chip;
forming a first conductive layer on the photo resist layer and the micro holes;
removing the photo resist layer;
forming a photosensitive polymer layer on the surface of the chip, wherein the photosensitive polymer layer covers the pads and fills each of the micro holes;
patterning the photosensitive polymer layer to form a plurality of polymer bumps;
forming a second conductive layer on a surface of each of the polymer bumps, wherein the second conductive layer is electrically connected to the pad; and
thinning the chip, so that one end of each of the polymer bumps protrudes from the chip.

23. The method according to claim 22, wherein the micro holes are formed by a drilling process or a dry etching process.

24. The method according to claim 22, wherein the first conductive layer and the second conductive layer are made of a biocompatible conductive material or a non-metallic conductive material.

25. The method according to claim 24, wherein the biocompatible conductive material is selected from the group consisting of titanium, gold, platinum, and their oxides.

26. The method according to claim 24, wherein the non-metallic conductive material is selected from the group consisting of iridium oxide and graphite.

27. A method for fabricating an artificial retina chip module, comprising:
providing a signal processing chip having a plurality of pads disposed on a surface of the signal processing chip;
forming a photo resist layer on the surface of the signal processing chip for covering the pads;
forming a plurality of micro holes passing through the photo resist layer and the pads, and extending inside the signal processing chip;
forming a first conductive layer on the photo resist layer and the micro holes;
removing the photo resist layer;
forming a photosensitive polymer layer on the surface of the chip, wherein the photosensitive polymer layer covers the pads and fills each of the micro holes;
patterning the photosensitive polymer layer to form a plurality of polymer bumps;
forming a second conductive layer on a surface of each of the polymer bumps, the second conductive layer being electrically connected to the pad;
thinning the signal processing chip, so that one end of each of the polymer bumps protrudes from the chip; and
providing a photodiode array chip and electrically connecting the signal processing chip with the photodiode array chip through the polymer bumps.

28. The method according to claim 27, wherein the micro holes are formed by a drilling process or a dry etching process.

29. The method according to claim 27, wherein the first conductive layer and the second conductive layer are made of a biocompatible conductive material or a non-metallic conductive material.

30. The method according to claim 29, wherein the biocompatible conductive material is selected from the group consisting of titanium, gold, platinum, and their oxides.

31. The method according to claim 29, wherein the non-metallic conductive material is selected from the group consisting of iridium oxide and graphite.

32. The method according to claim 27, wherein the heights of the polymer bumps vary according to a real curvature of the retina to be treated.

33. The method according to claim 27, further comprising forming a biocompatible polymer layer at a back surface of the signal processing chip, wherein the biocompatible polymer layer exposes the polymer bumps connected to the retina to be treated.

34. The method according to claim 27, further comprising forming a biocompatible polymer material, wherein the biocompatible polymer material covers the signal processing chip, the polymer bumps, and the photodiode array chip, and exposes the polymer bumps connected to the retina to be treated.
